(19) United States
(12) Patent Application Publication
(10) Pub. No.: US 2005/0171568 A1
(43) Pub. Date: Aug. 4, 2005

(54) Catheter and guidewire exchange system with improved catheter design

(76) Inventor: Niall Duffy, Tuam (IE)

Correspondence Address: MEDTRONIC VASCULAR, INC.;IP LEGAL DEPARTMENT, 3576 UNOCAL PLACE, SANTA ROSA, CA, 95403, US

(21) Appl. No.: 10/769,582

(22) Filed: Jan. 30, 2004

(51) Int. Cl. A61M 29/00

(52) U.S. Cl. 606/191; 606/194

(57) ABSTRACT

A catheter guiding assembly includes a catheter and a catheter advancing and retracting apparatus. The catheter includes an elongate shaft having an exterior surface, a proximal end, and a distal end; a first lumen extending through the shaft and sized to receive a guidewire; and a guideway enabling transverse access from the shaft exterior surface to the first lumen. The catheter advancing and retracting apparatus comprises an elongate housing having a proximal end and a distal end; an opening formed through the housing and adapted to receive the catheter; a wheel port formed in the housing and in communication with the opening; and a wheel secured in the wheel port and sized to radially extend into the catheter guideway.

### TECHNICAL FIELD

The present invention generally relates to catheters used in the vascular system, and more particularly relates to systems for facilitating exchange of such catheters and associated guidewires, and for using such catheters and guidewires to access selected sites within a patient.

### BACKGROUND

Catheters are inserted into various locations within a patient for a wide variety of purposes and medical procedures. Catheter insertion typically requires the use of a guidewire, particularly when the catheter carries a stent or other relatively bulky therapeutic device. The guidewire may be inserted into a patient's vasculature through the skin, and advanced to the treatment location. Alternatively, the guidewire and the delivery catheter may be advanced together, with the guidewire protruding from the catheter distal end. In either case, the guidewire guides the delivery catheter to the treatment location.

A guiding catheter is utilized in situations involving treatment of small diameter vessels that are remotely located relative to the catheter entry point. During a percutaneous transluminal coronary angioplasty (PTCA), the guiding catheter is typically inserted into a large artery near the patient's groin, and then advanced toward the heart until it reaches the diseased coronary artery ostium. The guiding catheter provides a tubular conduit through which smaller catheters and guidewires can be passed from outside the patient to the vessel being treated.

There are three general types of catheters: “over the wire” (OTW) catheters, “rapid exchange” (RX) or single operator catheters and “fixed wire” (FW) or “balloon on a wire” catheters. An OTW catheter includes a guidewire lumen that extends the entire catheter length. The guidewire is disposed entirely within the catheter guidewire lumen except for the distal and proximal guidewire portions, which extend beyond the distal and proximal catheter ends, respectively. An OTW catheter typically has a coaxial catheter construction, wherein two hollow tubes are nested together. FIG. 1 is a cross-sectional view of one type of OTW catheter 100, illustrated inside of a guiding catheter 105. The OTW catheter shaft 100 includes two coaxial hollow tubes 101, 102 that are nested together. An inner tube lumen 117 is adapted to slidably receive a guidewire 115, and an annular luminal space 119 formed between the outer and inner tubes 101, 102 is adapted for inflation/deflation fluid transportation. An alternative “multi-lumen” OTW catheter construction, a cross section of which is depicted in FIG. 2, has an elongate shaft 100′ made from a single extruded tube having two lumens 117′, 119′ formed side-by-side. OTW catheters that include both multi-lumen segments and coaxial segments are also known.

OTW catheters have many advantageous features traceable to the presence of a full-length guidewire lumen. Some of these features are good stiffness and pushability for readily advancing the catheter through tortuous vasculature and across tight stenoses. The full-length guidewire lumen can be used to transport radiocontrast dye to the stenosed artery, to make pressure measurements, to infuse drugs, and for other therapies. The full-length guidewire lumen also permits removal and replacement of a guidewire in an indwelling catheter, as it is sometimes desirable to exchange one guidewire for another guidewire having a different stiffness or tip shape.

OTW catheters do suffer some shortcomings, however. For example, it often is necessary to exchange one indwelling catheter for another catheter. In order to maintain a guidewire position while withdrawing a catheter, the guidewire must be gripped at its proximal end to prevent it from being pulled out of the blood vessel along with the catheter. For example, a PTCA catheter is sometimes longer than the proximal portion of the standard guidewire that protrudes out of the patient. Therefore, exchanging an OTW PTCA catheter requires an exchange guidewire of about 300 cm in length, whereas a standard guidewire is about 165 cm long.

In one type of OTW catheter exchange, the standard length guidewire is first removed from the indwelling catheter lumen. Then, a longer exchange guidewire is passed through the catheter to replace the original wire. Next, while holding the exchange guidewire by its proximal end to control its position in the patient, the catheter is withdrawn proximally from the blood vessel over the exchange guidewire. After the first catheter has been removed, another OTW catheter is threaded onto the exchange guidewire proximal end and is advanced along the exchange guidewire, through the guiding catheter, and into the patient's blood vessels until the catheter distal end is at the desired location. The exchange guidewire may be left in place or it may be exchanged for a shorter, conventional-length guidewire. In an alternative catheter exchange procedure, the initial guidewire length may be extended by way of a guidewire extension apparatus. Regardless of which exchange process is used, the very long exchange guidewire is awkward to handle, thus requiring at least two operators to perform the procedure.

Catheter designs have been developed in an attempt to eliminate the need for guidewire extensions or exchange guidewires. One such catheter design is the RX catheter, which is formed with the guidewire located outside of the catheter due to a short guidewire lumen that extends through a comparatively short distal catheter segment. The guidewire proximal exit port is typically located about 5 cm to about 30 cm proximal to catheter distal end. During use, the guidewire is initially placed in the patient's vascular system, and the catheter distal segment is then threaded onto the guidewire. The catheter can be advanced alongside the guidewire with its distal segment being attached to and guided along the guidewire. The catheter can be removed and exchanged for another RX catheter without the need for a relatively long exchange guidewire and without withdrawing the initially placed guidewire.

Although the RX catheter system has many advantages, some difficulties arise during use of the catheter. First, without a full-length guidewire lumen, the catheter proximal shaft is not interrelated with the guidewire. Consequently, the catheter distal end can not be easily pushed through tight stenoses or tortuous blood vessels. Ease in distal end pushability has consequently made the OTW catheters remain a practical choice for many procedures. Improved RX catheters have incorporated stiff, metal proximal shafts, and axial overlap between the shaft and the guidewire lumen to overcome pushability deficiencies. However, the non-aligned or offset arrangement between the guidewire and the shaft can cause shaft buckling as the catheter distal end is pushed along. Another difficulty associated with RX catheters a lack of ability to exchange guidewires in an indwelling RX catheter. A guidewire can be withdrawn from the proximal guidewire port, sometimes unintentionally, thereby derailing the indwelling catheter. Neither the original guidewire nor a replacement guidewire can be directed back into the catheter proximal guidewire port because the port is remotely hidden in the guiding catheter within the patient. Also, the lack of a full-length guidewire lumen deprives the clinician of an additional lumen that may be used for other purposes, such as pressure measurement, contrast dye injection, or drug infusion. A further difficulty associated with RX catheters lies in the relatively short guidewire lumen. Sometimes the guidewire lumen is so short that the proximal guidewire ports exits from the guiding catheter distal end, exposing the guidewire. Guidewire exposure presents a risk of what is called the “cheese cutter effect” which is damage to the curved artery delicate inner surface as straightening tension is applied to the exposed guidewire during push-pull maneuvers to advance the catheter. The short-lumen RX device also presents an increased risk of guidewire entanglement in procedures that use multiple guidewires because the guidewires are exposed within the blood vessel. Furthermore, the exposed unprotected portion of the guidewire can become kinked or tangled within the patient, adding complications to the procedure.

Another type of catheter designed to eliminate the need for guidewire extensions or exchange wires is sometimes referred to as an over-the-wire/short wire (OTW/SW) catheter. A cross section of one type of OTW/SW catheter is depicted in FIG. 3. The catheter 300 includes a catheter shaft 130 having a cut 128 that extends longitudinally between the catheter proximal end and the catheter distal end, and also extends radially from the catheter shaft outer surface to the guidewire lumen 126. A luminal space 122 for fluid or gas delivery, for example, is included in a side-by-side arrangement with the guidewire lumen 126. FIG. 4 is a sectional view of a guiding tool 150 that is used during catheter insertion. The guiding tool 150 is coupled to the catheter shaft 130 and includes a spreader member 154 that functions to open the cut 128 so the guidewire 115 may extend transversely into or out of the cut 128 at any location along the catheter 300. A guiding tube 148 is provided to assist in directing the guidewire 115 into the guidewire lumen 126. By moving the catheter 300 relative to the guiding tool, the effective over-the-wire portion of the OTW/SW catheter is adjustable.

When using the OTW/SW catheter, the guidewire is maneuvered through the patient's vascular system with the guidewire distal end positioned across the treatment site. With the guide member positioned near the catheter distal end, the guidewire proximal end is threaded into the guidewire lumen opening at the catheter distal end and through the guide member so that the guidewire proximal end protrudes out of the guiding tool proximal end. By fixing the guide tool and the guidewire proximal end relative to one another, the catheter may be transported over the guidewire by advancing the catheter toward the guide tool. As the catheter advances through the guide tool and into the patient's vasculature, the guidewire lumen envelops the guidewire.

By reversing the above operation, an indwelling OTW/SW catheter may be exchanged with another OTW/SW catheter. The indwelling catheter may be removed by withdrawing the catheter proximal end from the patient while securing the guidewire proximal end and the guide tool in a fixed position. When the catheter is withdrawn to the extent that the distal end reaches the guide tool, the length of the catheter distal portion that remains around the guidewire is sufficiently short to draw the catheter over the guidewire proximal end without releasing control of the guidewire or disturbing its position within the patient. After the catheter is removed, another OTW/SW catheter may be threaded onto the guidewire and advanced in the same manner described above. The OTW/SW catheter also allows a guidewire to be removed from an indwelling catheter and reinserted or exchanged without having to withdraw the catheter from the patient. Thus, the OTW/SW catheter overcomes many difficulties associated with the OTW and RX catheters.

Recently efforts have been directed toward minimizing the complexity and size of the guide tool that is used with the OTW/SW catheter. The catheter with the side-by-side lumen arrangement has a relatively large outer diameter, and requires a correspondingly large guide tool to accommodate the catheter. Accordingly, it is desirable to provide an OTW/SW catheter that has a smaller outer diameter, so that the guide tool can have a correspondingly smaller inner diameter, and consequently a smaller overall size. In addition, it is desirable to provide a guide tool that is less complex than the current guide tool, allowing the user to quickly troubleshoot, prevent, and correct any difficulties that may occur during guide tool operation. Furthermore, other desirable features and characteristics of the present invention will become apparent from the subsequent detailed description and the appended claims, taken in conjunction with the accompanying drawings and the foregoing technical field and background.

### BRIEF SUMMARY

An assembly is provided for guiding a catheter. The assembly comprises a catheter and a catheter advancing and retracting apparatus. The catheter comprises an elongate shaft having an exterior surface, a proximal end, and a distal end; a first lumen extending through the shaft from the shaft proximal end to the shaft distal end, and sized to receive a guidewire; and a guideway extending from the shaft proximal end to the shaft distal end, and enabling transverse access from the shaft exterior surface to the first lumen. The catheter advancing and retracting apparatus comprises an elongate housing having a proximal end and a distal end; an opening formed through the housing from the housing proximal end to the housing distal end, and adapted to receive the catheter; a wheel port formed in the housing between the housing proximal and distal ends and in communication with the opening; and a wheel secured in the wheel port and sized to radially extend into the catheter guideway when the catheter is received in the opening.

An apparatus is also provided for advancing and retracting a catheter and guidewire in a patient. The apparatus comprises an elongate housing having a proximal end and a distal end; a opening formed through the housing from the proximal end to the distal end and adapted to house the catheter and guidewire; a wheel port formed in the housing between the proximal and distal ends and in communication with the opening; and a wheel secured in the wheel port and radially extending into the opening to engage with the catheter.

A catheter is also provided. The catheter comprises an elongate shaft having an exterior surface, a proximal end, and a distal end; a first lumen extending through the shaft from the shaft proximal end to the shaft distal end, and sized to receive a guidewire; and a guideway extending from the shaft proximal end to the shaft distal end, and enabling transverse access from the shaft exterior surface to the first lumen.

### BRIEF DESCRIPTION OF THE DRAWINGS

The present invention will hereinafter be described in conjunction with the following drawing figures, wherein like numerals denote like elements, and

FIG. 1 is a cross-sectional view of a contemporary over-the-wire catheter;

FIG. 2 is a cross-sectional view of a contemporary multi-lumen over-the-wire catheter;

FIG. 3 is a cross section of a contemporary over-the-wire/short wire catheter;

FIG. 4 is a sectional view of a contemporary catheter guiding tool that is used during catheter insertion;

FIG. 5 is a perspective view of a catheter guiding assembly according to an embodiment of the present invention;

FIG. 6 is a perspective view of a catheter advancement/retraction wheel according to an embodiment of the present invention;

FIG. 7 is a perspective view of the catheter advancement/retraction wheel frictionally engaged with a novel catheter according to an embodiment of the present invention;

FIG. 8 is a sectional view of a catheter guiding assembly including the catheter advancement/retraction wheel frictionally engaged with the catheter according to an embodiment of the present invention; and

FIG. 9 is a sectional view of a catheter according to an embodiment of the present invention, including a nearly coaxially arranged guidewire lumen and inflation lumen.

FIG. 10 is a sectional view of a catheter with a guidewire being removed from a guidewire lumen using a scoop according to an embodiment of the present invention;

FIG. 11 is a perspective view of a guidewire removal tool according to an embodiment of the present invention; and

FIG. 12 is a sectional view of a catheter with the guidewire removal tool depicted in FIG. 11 inserted into a guidewire lumen according to an embodiment of the present invention.

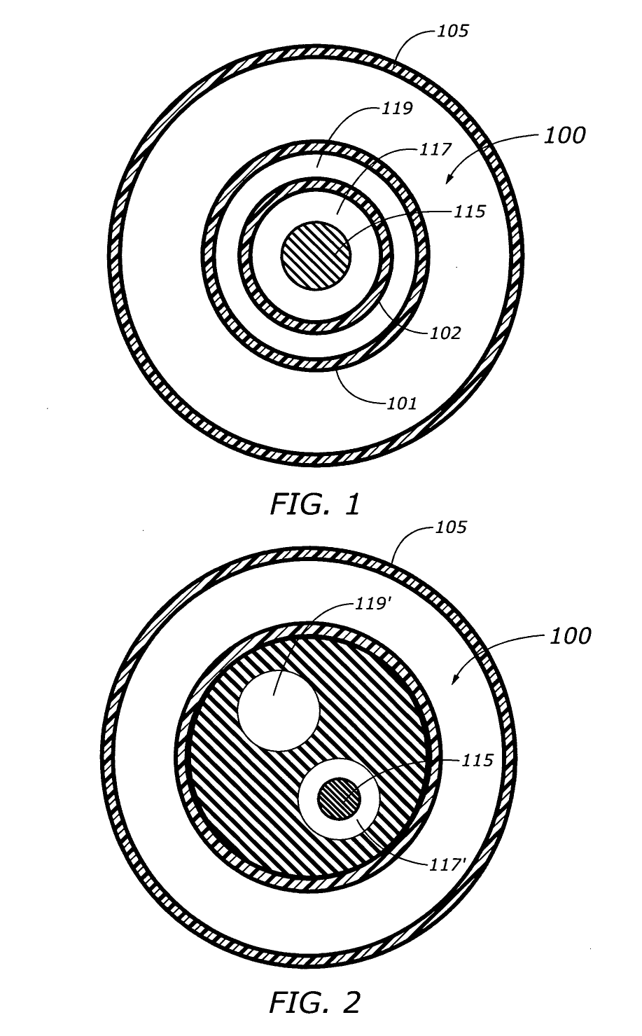

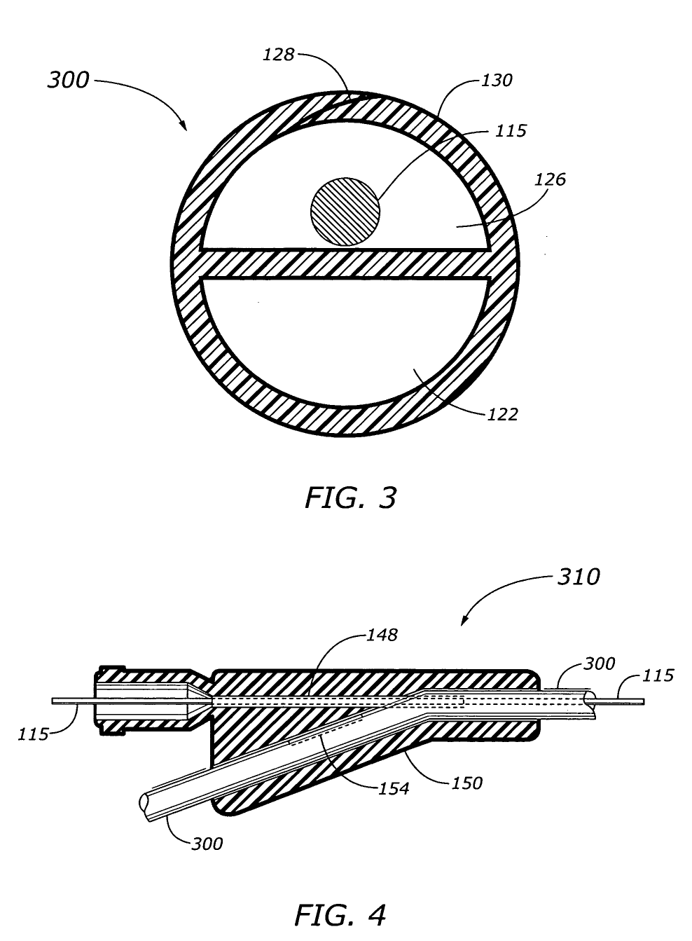

### DETAILED DESCRIPTION

The following detailed description is merely exemplary in nature and is not intended to limit the invention or the application and uses of the invention. Furthermore, there is no intention to be bound by any expressed or implied theory presented in the preceding technical field, background, brief summary or the following detailed description.

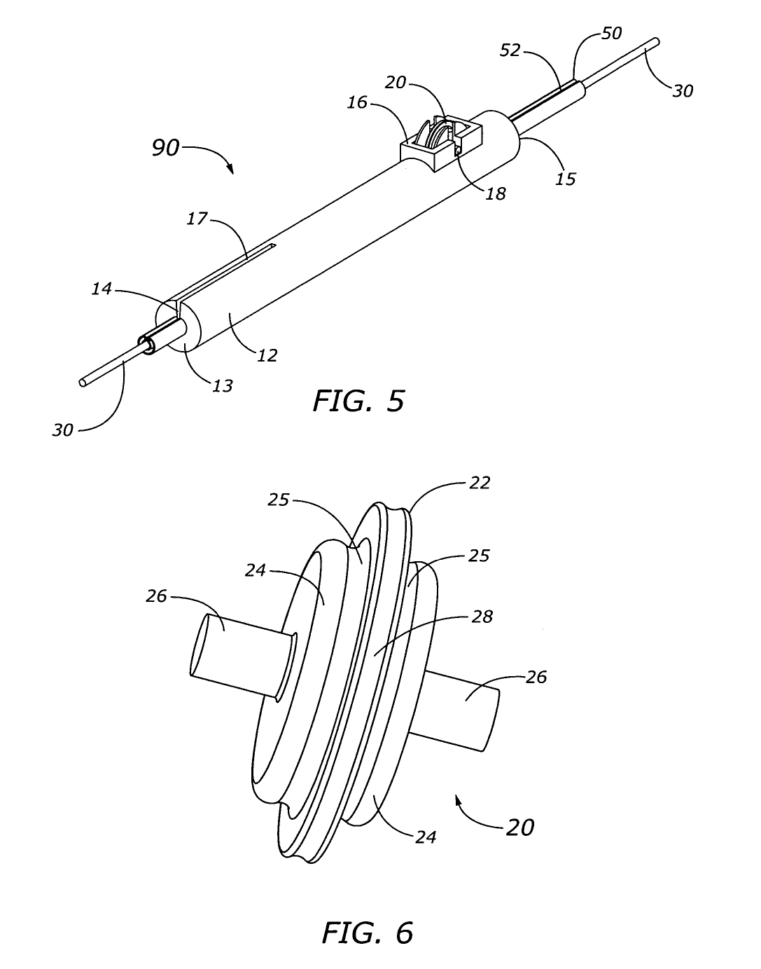

The present invention includes a catheter construction with a relatively small outer diameter, and tools for advancing and retracting the catheter and for coupling and decoupling the catheter with a guidewire. FIG. 5 is a perspective view of a catheter guiding assembly 90 according to an exemplary embodiment of the invention. The assembly includes a housing 12 that is depicted in conjunction with a catheter 50. The housing 12 has a proximal end 13 and a distal end 15, with the distal end 15 being closest to the patient and the proximal end 13 being farthest away from the patient. The catheter 50 travels through a central opening 14 as it is advanced into or retracted from a location within a patient. The catheter's advancement or retraction is manipulated by rotating a wheel 20 that is secured in a wheel receiving body or port 16 that is part of the housing 12. The port 16 is in communication with the central opening 14 and consequently allows the wheel 20 to radially extend into the central opening 14 and frictionally engage with the catheter 50. As the wheel 20 rotates, it pushes a guidewire 30 into a guideway 52 formed in the catheter 50.

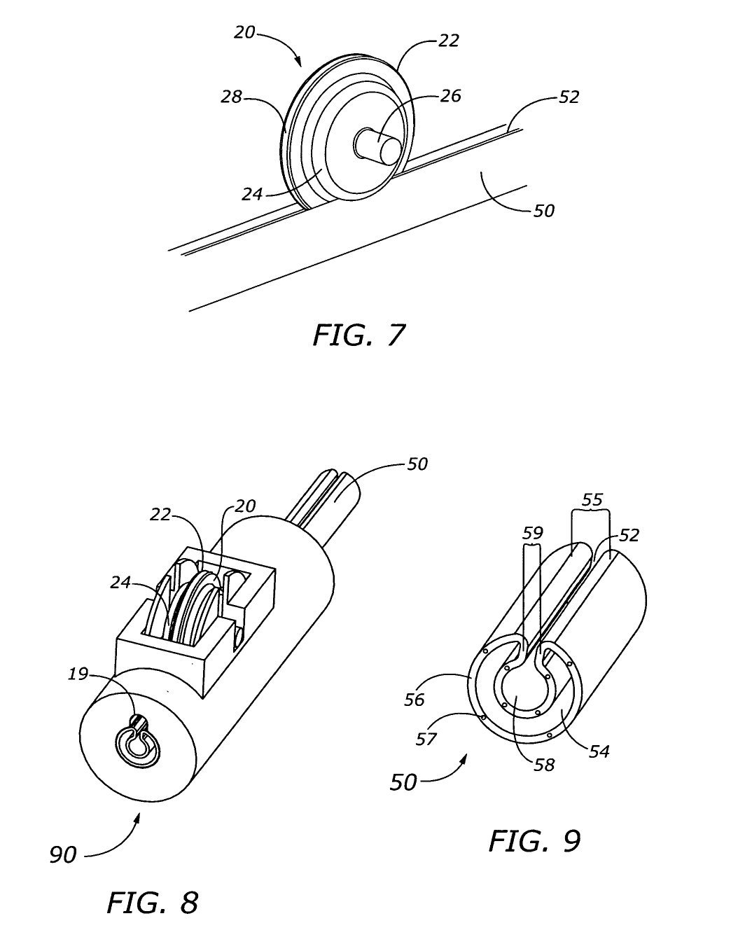

FIG. 6 is a perspective view of the wheel 20 according to one embodiment of the invention. The wheel 20 includes an axle 26 that is receivable into slots 18 formed in the wheel receiving port 16. The entire wheel 20 rotates as the catheter 50 is advanced and retracted. With the axle secured and rotating with little friction in the slots 18, there is little resistance to catheter advancement and retraction. The slots 18 may each include a clip or other device to hold the wheel 20 in place and thereby prevent the wheel from disengaging with the catheter 50. Approximately at the center of the wheel width, a thin, large diameter portion 22 extends radially from two flanking smaller diameter portions 24. The large diameter portion 22 is used to force open the catheter guideway 52, and also to push the guidewire 30 into the catheter guideway 52. The smaller diameter portions 24 are frictionally engaged with the catheter 50 on each side of the catheter guideway 52 and are used, together with the large diameter portion 22, to advance and retract the catheter 50 as the wheel 20 rotates. FIG. 7 is a perspective view of the wheel 20 and the catheter 50 positioned with the large diameter wheel portion 22 in the catheter guideway 52 and the small diameter portions frictionally engaged with the catheter 50. FIG. 8 is a sectional view of the catheter guiding assembly 90 proximal to the wheel 20, with the wheel and catheter 50 in the same arrangement depicted in FIG. 7. In an exemplary embodiment of the invention, the large diameter portion 22 has a concave outer surface 28 to prevent the guidewire 30 from moving laterally as the guidewire 30 is pushed into the catheter guideway 52. Further, the opening 14 in the guiding assembly 90 is continuous with an arched guidewire passageway 19 proximal to the wheel 20 to contain and laterally support the guidewire 30 as it moves between the guiding assembly proximal end 13 and the wheel 20 inside the guiding assembly 90.

The catheter guiding assembly 90, including the wheel 20, is made from blends of polyamides and polyolefins in an exemplary embodiment of the invention. Other exemplary materials include ceramics, metals such as stainless steel, and other polymers such as polyamides and liquid crystal polymers. Lubrication additives such as polyethylene micro-powders, fluoropolymers, silicone-based oils, fluoro-ether oils, molybdenum disulphide, and polyethylene oxide may be included. Reinforcing additives such as nano-clays, graphite, carbon fibers, glass fibers, polyesters, polyketones, polyimides, polysulphones, polyoxymethylenes, polyolefms, cross-linked polyolefins may also be included, along with compatibilizers based on polyolefins, such as grafted polyolefins, ceramics, and metals.

Turning now to the catheter 50 used with the guiding assembly 90, FIG. 9 is a sectional view of the catheter 50, including a nearly coaxially arranged guidewire lumen 58 and inflation lumen 54. Both lumens 54, 58 are formed from a single continuous shaft wall 56 that may be formed from suitable biomedical grade materials such as polyethylene, cross-linked polyethylene, polyolefins, polyamides, blends of polyamides and polyolefins, fluoropolymers, polyesters, polyketones, polyimides, polysulphones, polyoxymethylenes, and compatibilizers based on polyolefins, including grafted polyolefins, and other comparable materials. A lubrication additive may also be used with any polymer and may include polyethylene micro-powders, fluoropolymers, silicone based oils, fluoro-ether oils, molybdenum disulphide and polyethylene oxide. Additionally, a reinforcing additive may be used such as nano-clays, graphite, carbon fibers, glass fibers, and polymeric fibers. The shaft wall 56 that defines the entire inflation lumen 58 is depicted in FIG. 9 as having a substantially uniform thickness, which may simplify the catheter manufacturing process and reduce the associated costs. However, the shaft wall 56 may also be formed with a varying thickness to provide strength to the catheter 50 as needed.

Both the guidewire lumen 58 and the inflation lumen 54 extend from the catheter distal end to the proximal end in an exemplary embodiment of the invention. The generally c-shaped, nearly annular inflation lumen 54 almost entirely surrounds the guidewire lumen 58. A guideway 52 interrupts the inflation lumen's annular shape and prevents the inflation lumen 54 from forming a complete ring. However, the nearly annular inflation lumen 54 has a large cross sectional area for rapid gas transport. Further, the shaft inner diameter that defines the guidewire lumen 58 is at least 0.015 inch, which is wide enough to permit free movement of contemporary guidewires, which are typically have an outer diameter of about 0.014 inch. The shaft has a substantially uniform outer diameter of about 1 mm (≈0.039 inch) in an exemplary embodiment.

Stiffening wires 57 may be incorporated into the lumen walls to strengthen the catheter and maintain its shape. The wires 57 may be incorporated into the catheter 50 in several ways. The wires 57 can be placed in the inflation lumen and attached to the lumen walls, or the shafts can be molded around the wires 57, as illustrated in FIG. 9. Instead of or in addition to stiffening wires 57, a stiffening member in the form of a substantially c-shaped metal strip may be disposed inside the inflation lumen and lining the lumen walls. The stiffening member may have another shape to adapt to the catheter shape and use, but the stiffening member should not impede gas flow through the inflation lumen.

The guideway 52 is defined by approximately parallel wall segments 59 that are adapted to be flexibly spaced apart to provide transverse access for the guidewire to enter and exit the guidewire lumen 58. When the catheter 100 is tightened in a Y-adapter, the wall segments 59 will rest flatly against each other, thereby preventing back-bleed and also sealing the guideway 52 and reducing or eliminating any clearance around the guidewire. Also, if a physician draws a vacuum on the Y-adapter to draw blood from the patient using an inflation syringe, there is a danger that a gas may be drawn into the Y-adapter through any gap that exists between the guidewire 30 and the catheter shaft wall 56. With the wall segments 59 sealing the guideway 52, and eliminating space around the guidewire 30, gas aspiration is also improved.

In an exemplary embodiment of the invention, a guidewire entrance 55 with a gradually curved contour joins the guideway 52 and the catheter outer diameter. The guidewire entrance 55 further prevents the guidewire 30 from moving laterally and thereby impeding its insertion into the guideway 52. In a further exemplary embodiment, the wheel smaller diameter portions 24 that flank the large diameter portion 22 have concave surfaces 25 that are rounded to match the catheter's curved guidewire entrance 55 and thereby improve the frictional engagement between the wheel 20 and the catheter 50.

The operation of the catheter 50 and the guiding assembly 90 will now be described. After the guidewire 30 is inserted into the patient, the guidewire 30 can be combined with the catheter by backloading the catheter 50 onto the guidewire 30. Using a backloading process, the guidewire proximal end is threaded through the guiding assembly opening 14, and then threaded into the catheter guidewire lumen 58 until the guidewire proximal end either exits the catheter proximal end or reaches a proximal position of the catheter 50 relative to the wheel 20, at which point the guidewire 30 can be removed from the guidewire lumen 58 using a suitable tool. With the guidewire 30 removed from the guidewire lumen, the guidewire 30 can be held in place as the catheter 50 is advanced through the guiding assembly 90 and into the patient. As the catheter 50 advances, the wheel pushes the stationary guidewire 30 into the guidewire lumen 58.

If a guidewire exchange is required, the physician or other user can simply pull out the guidewire 30 with the catheter 50 remaining stationary. A new guidewire can be loaded into the proximal guidewire lumen 58 and threaded through the catheter 50. The distal portion of the guidewire 30 is typically flexible and difficult to insert into the guidewire lumen 58. Consequently, the distal end of the new guidewire 30 can be positioned above the guideway 59, and then, with the catheter 50 and guidewire 30 kept stationary, the guiding assembly 90 can be moved proximally over the guidewire until the wheel 20 pushes the guidewire 30 into the guidewire lumen 58. Once the guidewire flexible distal portion is inside the guidewire lumen 58, the guidewire can be advanced by pushing the guidewire 30 through the catheter 50, and the guiding assembly 90 can be returned to a position closer to the patient if necessary.

If a catheter exchange is required, the physician or other user holds the guidewire 30 in place and retracts the catheter proximally by rotating the wheel 20. After the catheter is removed, the replacement catheter is installed using the process described above.

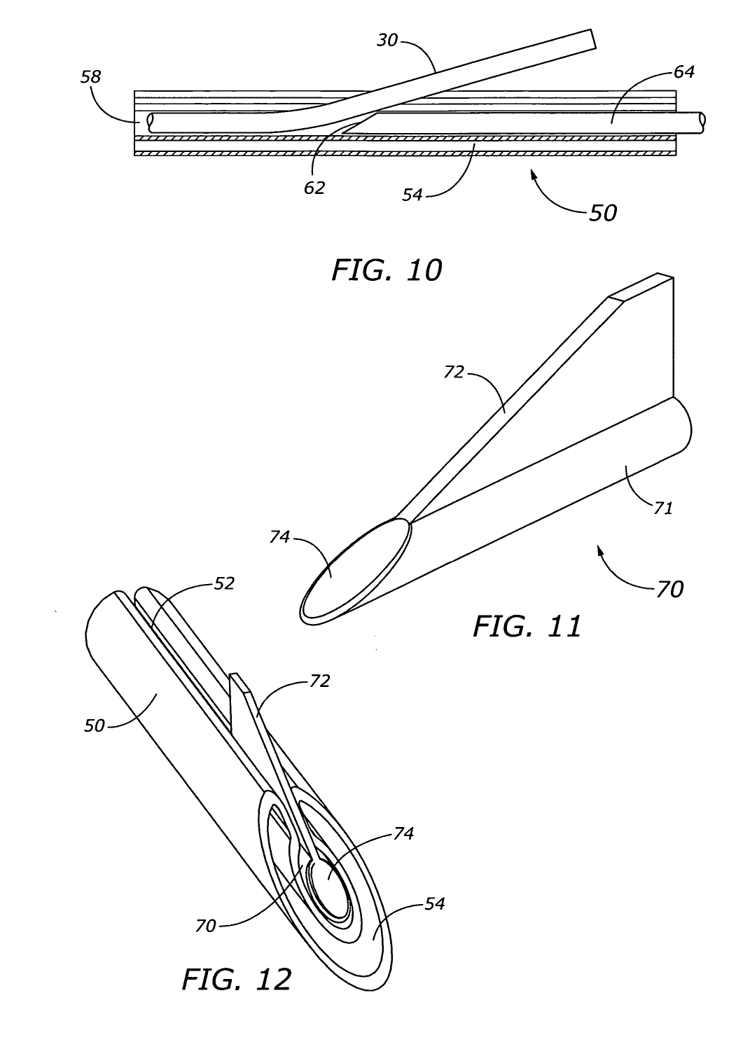

As mentioned above, it is typically necessary to remove at least a portion of the guidewire 30 from the proximal catheter shaft. A suitable tool may be utilized in order to quickly remove the guidewire 30. FIG. 10 is a sectional view of the catheter 50 with the guidewire 30 partially disposed in the guidewire lumen 58, and partially removed from the guidewire lumen 58 using a guidewire removal tool 60 according to one embodiment of the invention. The tool 60 includes a substantially cylindrical main body 64, and a chamfered leading edge 62 that can be a flat beveled surface, a concave or grooved surface, a v-shaped surface, or other suitable surface that can raise the guidewire 30 out of the guidewire lumen 58.

The main body 64 is sized to have a diameter that approximates that of the guidewire 30 so the tool 60 can effectively raise the guidewire 30 and force it out from the guidewire lumen 58. The main body 64 may have a slightly larger diameter than the guidewire 30 as long as the diameter is not larger than the guidewire lumen inner diameter or large enough to create enough friction to prevent the tool 60 from extending a significant distance into the guidewire lumen 58. In an exemplary embodiment of the invention, the main body 64 has a substantially uniform diameter ranging between about 0.016 inch and about 0.017 inch, and is used with a guidewire 30 that has a diameter of about 0.017 inch.

In an exemplary embodiment of the invention, the tool 60 is a rigid body and is formed entirely from a metallic material. The strong and rigid metal provides the advantages of ease in placing the tool 60 in a desired location and thereafter manipulating the tool to raise the guidewire 60. If the tool will be distally extended a significant distance into the catheter 50 then the metallic material can be somewhat bendable although the tool 60 should be rigid enough to easily manipulate the leading edge when holding the tool 60 from a from an upstream or proximal point. The tool 60 can be formed from a wire mandrel and can be as long or as rigid as necessary to perform the desired function. One advantage of a substantially elongated and rigid tool 60 is its ability to perform a stiffening function for at least some catheter length that is proximal to the catheter guiding assembly 100. For instance, without the tool 60 inserted into the guidewire lumen 58, the catheter is advanced by grasping the catheter 50 a short distance from the guiding assembly 90 and pushing the catheter into the guiding assembly. With a long and rigid tool 60 inserted into the guidewire lumen 58, the advancing force can be applied to the catheter 50 much farther away from the guiding assembly 90, and consequently a longer catheter length can be advanced for each push.

FIG. 11 is a perspective view of another exemplary embodiment of a guidewire removal tool 70. The tool 70 includes a substantially cylindrical main body 71, and a chamfered leading edge 74 that can be a flat beveled surface, a concave or grooved surface, a v-shaped surface, or other suitable surface that can raise the guidewire 30 out of the guidewire lumen 58, and can have the characteristics as the cylindrical main body 64 described above. The tool 70 also includes a fin 72 that interacts with the guidewire lumen 58 in the manner illustrated in FIG. 12, which is a sectional view of the catheter 50 with the guidewire removal tool 70 inserted in the guidewire lumen 58. Returning to FIG. 5, the catheter guiding assembly 100 can include a receiving slot 17 through which the fin 72 can protrude as the tool 70 is advanced into the guiding assembly 90 from the proximal end 13. In an exemplary embodiment of the invention, the fin 72 is stabilized by the slot 17, which also fixes the tool 70 in the guiding assembly 90. In another exemplary embodiment, the fin 72 provides a handle for the physician or other user to push the tool into the catheter guiding assembly 90. More particularly, the fin 72 protrudes from the receiving slot 17 and enables the physician or other user to move the tool 70 close to the wheel 20 and thereby remove the guidewire 30 from the guidewire lumen 58 near the guiding assembly distal end 15.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing the exemplary embodiment or exemplary embodiments. It should be understood that various changes can be made in the function and arrangement of elements without departing from the scope of the invention as set forth in the appended claims and the legal equivalents thereof.

1. A catheter guiding assembly, comprising:
a catheter, comprising:
an elongate shaft having an exterior surface, a proximal end, and a distal end,
a first lumen extending through the shaft from the shaft proximal end to the shaft distal end, and sized to receive a guidewire, and
a guideway extending from the shaft proximal end to the shaft distal end, and enabling transverse access from the shaft exterior surface to the first lumen; and
a catheter advancing and retracting apparatus, comprising:
an elongate housing having a proximal end and a distal end,
an opening formed through the housing from the housing proximal end to the housing distal end and adapted to receive the catheter,
a wheel port formed in the housing between the housing proximal and distal ends and in communication with the opening, and
a wheel secured in the wheel port and sized to radially extend into the catheter guideway when the catheter is received in the opening.

2. The catheter guiding assembly according to claim 1, wherein the catheter further comprises a second lumen extending through the shaft from the shaft proximal end to the shaft distal end, and having a nearly annular cross sectional area that almost entirely surrounds the first lumen.

3. The catheter guiding assembly according to claim 2, wherein the first lumen and the second lumen are both formed from a single wall having a substantially uniform thickness.

4. The catheter guiding assembly according to claim 3, wherein the single wall includes two approximately parallel segments that together define the catheter guideway.

5. The catheter guiding assembly according to claim 4, wherein the two parallel segments are adapted to be flexibly spaced apart when receiving the wheel.

6. The catheter guiding assembly according to claim 1, wherein the wheel port includes slots, and the wheel includes an axle that is rotatably secured in the slots.

7. The catheter guiding assembly according to claim 1, wherein the wheel includes an axle, a large diameter portion and two small diameter portions flanking the large diameter portion.

8. The catheter guiding assembly according to claim 7, wherein the large diameter portion has a circumferential concave surface.

9. The catheter guiding assembly according to claim 7, wherein each of the small diameter portions has a circumferential concave surface.

10. The catheter guiding assembly according to claim 7, wherein the large diameter portion is approximately centered between the small diameter portions.

11. The apparatus according to claim 1, wherein the wheel port is formed proximate to the housing distal end.

12. The apparatus according to claim 1, further comprising:
a guidewire removal tool, comprising a substantially cylindrical main body sized to be receivable by the first lumen and having a chamfered leading edge adapted to raise the guidewire out of the first lumen through the guideway.

13. An apparatus for advancing and retracting a catheter and guidewire in a patient, the apparatus comprising:
an elongate housing having a proximal end and a distal end;
a opening formed through the housing from the proximal end to the distal end and adapted to house the catheter and guidewire;
a wheel port formed in the housing between the proximal and distal ends and in communication with the opening; and
a wheel secured in the wheel port and radially extending into the opening to engage with the catheter.

14. The apparatus according to claim 13, wherein the wheel port includes slots, and the wheel includes an axle that is rotatably secured in the slots.

15. The apparatus according to claim 13, wherein the wheel includes an axle, a large diameter portion and two small diameter portions flanking the large diameter portion.

16. The apparatus according to claim 14, wherein the large diameter portion has a circumferential concave surface.

17. The apparatus according to claim 15, wherein each of the small diameter portions has a circumferential concave surface.

18. The apparatus according to claim 15, wherein the large diameter portion is approximately centered between the small diameter portions.

19. The apparatus according to claim 13, wherein the wheel port is formed proximate to the housing distal end.

20. A catheter, comprising:
an elongate shaft having an exterior surface, a proximal end, and a distal end;
a first lumen extending through the shaft from the shaft proximal end to the shaft distal end, and sized to receive a guidewire; and
a guideway extending from the shaft proximal end to the shaft distal end, and enabling transverse access from the shaft exterior surface to the first lumen.

21. The catheter according to claim 20, wherein the catheter further comprises a second lumen extending through the shaft from the shaft proximal end to the shaft distal end, and having a nearly annular cross sectional area that almost entirely surrounds the first lumen.

22. The catheter according to claim 21, wherein the first lumen and the second lumen are both formed from a single wall having a substantially uniform thickness.

23. The catheter according to claim 22, wherein the single wall includes two approximately parallel segments that together define the catheter guideway.

24. The catheter according to claim 23, wherein the two parallel segments are adapted to be flexibly spaced apart when receiving the wheel.
